(19) United States
(12) Patent Application Publication
(10) Pub. No.: US 2015/0258233 A1
(43) Pub. Date: Sep. 17, 2015

(54) DECONTAMINATION SYSTEM FOR A BUILDING OR ENCLOSURE

(71) Applicant: AEROCLAVE, LLC, Winter Park, FL (US)

(72) Inventors: Ronald D. Brown, Maitland, FL (US); David J. Robinson, Orlando, FL (US); Michael G. Quinoy, Maitland, FL (US); James S. Amrhein, Oviedo, FL (US)

(21) Appl. No.: 14/645,825

(22) Filed: Mar. 12, 2015

Related U.S. Application Data
(60) Provisional application No. 61/951,780, filed on Mar. 12, 2014.

(51) Int. Cl. A61L 2/20; A61L 2/24

(52) U.S. Cl. CPC A61L 2/20; A61L 2/24

(57) ABSTRACT

A decontamination system for a building comprises a disinfectant solution source with a disinfectant solution stored in a first room of the building. At least one nozzle is positioned in a second room of the building and the at least one nozzle is in fluid communication with the disinfectant source. One or more pumps are provided in fluid communication with each nozzle and the disinfectant solution source. In addition, a pressurized air supply is provided in fluid communication with each nozzle. One or more controllers is provided in signal communication with the one or more pumps and the pressurized air supply to activate the pump(s) and pressurized air supply to simultaneously deliver the disinfectant solution and pressurized air to one or more of the nozzles in a respective room. The system may also include a hub assembly with a nozzle and pump at the hub assembly to deliver disinfectant solution to the nozzle.

### CROSS-REFERENCE TO RELATED APPLICATIONS

This application claims the benefit of U.S. Provisional Application No. 61/951,780 filed Mar. 12, 2014, and incorporated herein by reference in its entirety.

### BACKGROUND OF THE INVENTION

The present invention relates to decontamination systems that generate an aerosol, mist, fog or spray from a liquid disinfectant solution to sanitize surfaces within a confined area. More specifically, the invention relates to such systems that provide for decontamination of a plurality of rooms within a building.

Surfaces and items within rooms are often exposed to harmful pathogens or infectious agents such as viruses, bacteria, fungus, parasites, etc. Liquids including chlorine dioxide, bleach, and formaldehyde may be used with cleaning items to sanitize a room; however, in some instances manual labor may be extremely inefficient for decontaminating an entire room or multiple surfaces. If the threat is significant enough the trained technicians performing a decontamination procedure may be required to wear HAZMAT suits for protection.

Systems have been developed that generate a decontaminant spray or fog applied to internal and external surfaces of a vehicle. One such system has been developed by AeroClave, LLC (Winter Park, Fla.) and is sold under the brand name PADS (Portable Asset Decontamination System). PADS is a chamber or enclosure that is large enough for a vehicle to enter. A disinfectant solution source and compressed air are provided in fluid communication to a plurality of nozzles spaced apart in the room. PADS also includes a control system to control pumps, valves and an air compressor to deliver the disinfectant solution to the nozzles at timed intervals to inject the disinfectant solution in a spray or fog form. A vehicle is driven in the chamber and the doors and/or windows are opened and the disinfectant in spray or fog form is distributed through the nozzles and throughout the room to disinfect the interior and exterior surfaces of the vehicle.

AeroClave has also developed a portable unit (the RDS 3110) that includes a container within which a reservoir containing a disinfectant solution, an air compressor, pumps and a controller are housed. The air compressor and pumps are within a housing in the container and the housing has a top cover that has portals to which nozzles can be fixed or removable. Alternatively, hoses with a nozzle at one end can be attached to the portals and the nozzles can be held by hand to apply disinfectant to surfaces of a vehicle or a room of a building, or the nozzles may be placed on a stand. As with PADS, the controller can be programmed to control delivery of the disinfectant solution for timed intervals to deliver a specified volume of disinfectant solution depending on the size of the surface area to be decontaminated or the volume of a room or interior of a vehicle to be decontaminated.

While the AeroClave PADS and RDS 3110 are effective in decontaminating a room or items within an enclosure, these products may not be as efficient in decontaminating multiple rooms of a building. The inventors of the subject invention are not aware of a commercially available automated system that employs one or more nozzles or disinfectant delivery devices in multiple rooms and a control system to deliver disinfectant solution to multiple rooms of a building from a single solution source.

### BRIEF DESCRIPTION OF THE INVENTION

The invention(s) disclosed herein may be particularly useful for sterilizing or decontaminating spaces and surfaces within rooms that are frequently exposed to harmful viruses, bacteria, chemicals, etc. Accordingly, the decontamination system may be beneficial for buildings in which a large number of people may work or move about during the course of a day. Such buildings may be any structure or enclosure including police/fire department buildings, hospitals, mass transit facilities, etc.; however, the subject invention is not limited to these types of buildings and may include a variety of different buildings.

In an embodiment, a decontamination system for a building comprises a disinfectant solution source with a disinfectant solution stored in a first room of the building. At least one disinfectant dispensing hub assembly is positioned in a second room of the building and the at least one hub assembly includes at least one nozzle in fluid communication with the disinfectant source. One or more pumps is provided in fluid communication with each nozzle and the disinfectant solution source, in addition, a pressurized air supply is provided in fluid communication with each nozzle. A plurality of fluid lines are connected to the disinfectant solution source and to the nozzles; and, a plurality of air lines connect the pressurized air supply to the nozzles. One or more controllers are provided in signal communication with the one or more pumps and the pressurized air supply to activate the pump(s) and pressurized air supply to simultaneously deliver the disinfectant solution and pressurized air to one or more of the nozzles in a respective room.

In another aspect, the invention may comprise one or motion detectors in a room having a hub assembly and the one or more controllers is configured to activate the one or more pumps to deliver disinfectant solution to the nozzles after a time duration during which the motion detector does not detect movement in the room.

In another aspect, the one or more controllers may be configured to initiate building functions related to a room to be decontaminated before and/or after disinfectant solution is delivered to the room. These building functions may include locking and/or unlocking a door to a room to be decontaminated and/or turning on and/or off HVAC functions to the room to be decontaminated.

In yet another aspect, the invention may comprise one or more sensors in fluid communication with a room to be decontaminated for monitoring levels of an active disinfectant of the disinfectant solution and the sensor is in signal communication with the one or more controllers transmitting signals indicative of a level of the active disinfectant in a room. In an embodiment, the sensor is in signal communication with a controller to transmit signals indicative of a level of the active disinfectant in the room and the controller is configured to generate a signal to an audible or visual indicator when the disinfectant is above or below a threshold concentration.

In another aspect, the invention may comprise a first pump operatively connected to the disinfectant solution source and may be in signal communication with the one or more controllers to deliver the disinfectant solution into the fluid lines. A hub assembly may include at least one second pump in fluid communication with the first pump and fluid lines to deliver the disinfectant solution to a nozzle at the hub assembly.

In another aspect, the one or more controllers may include a first controller in a room and a second controller at a hub assembly in another room, and an input device is associated with the first controller to input parameters associated with a decontamination operation. The first controller may be configured to, on command, activate the first pump, the pressurized air supply and the second controller, which in turn activates the at least one second pump.

### BRIEF DESCRIPTION OF THE DRAWINGS

FIG. 1 is a schematic representation of the decontamination system for a building in accordance with aspects of the invention.

FIG. 2 is an elevational view of a system control room in accordance with aspects of the invention.

FIG. 3 is an elevational view of a system control in accordance with aspects of the invention.

FIG. 4 is an elevational view a wall-mounted hub assembly in accordance with aspects of the invention.

FIG. 5 is an elevational view of the wall mounted hub assembly of FIG. 4 with a cover removed showing components of the hub assembly.

FIG. 6 is a perspective view of a ceiling hub assembly mounted to a ceiling frame member.

FIG. 7 is a perspective view of a pole mounted hub assembly that may be suspended from a ceiling of a room.

FIG. 8 is a perspective of a hub assembly and the components thereof in accordance with aspects of the invention.

FIGS. 9-15 are images of a touchscreen display of an input device in accordance with a decontamination operation of the subject invention.

### DETAILED DESCRIPTION OF THE INVENTION

A more particular description of the invention briefly described above will be rendered by reference to specific embodiments thereof that are illustrated in the appended drawings. Understanding that these drawings depict only typical embodiments of the invention and are not therefore to be considered to be limiting of its scope, the invention will be described and explained.

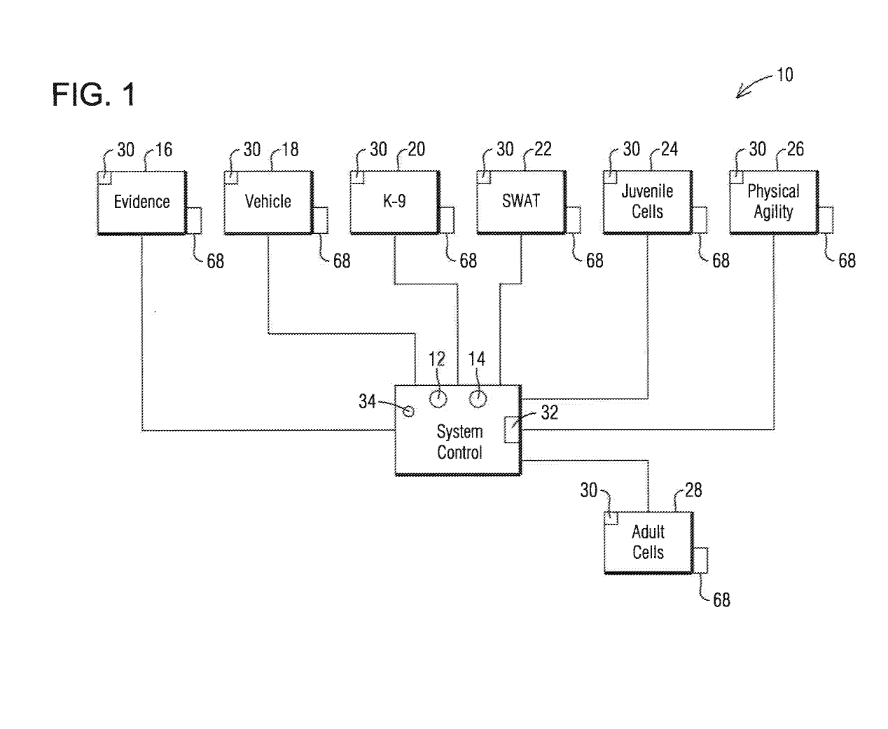

A decontamination system 10 for a building is schematically represented in FIG. 1 and includes a disinfectant solution source 12 and a pressurized air supply 14 in fluid communication with a plurality of nozzles 30, and each nozzle 30 is in a respective room of a building. By way of example the system may be used in connection with a police department building including an evidence room 16, a vehicle garage 18, a K-9 room 20, a S.W.A.T. room 22, juvenile holding cells 24, a physical agility room 26 and adult holding cells 28. However, the invention is not limited to these particular rooms, this number of rooms or this particular type of building. The decontamination system 10 may be used in any number of rooms for any type building. The term “building” as used herein is intended to mean a structure with a roof and walls that are used as a place for people to live, work, do activities, store things, etc. A building could also be used to house plants or animals.

The solution is preferably an aqueous disinfectant solution. Disinfectants that may be used to disinfect surfaces of an interior space may comprise hydrogen peroxide, chlorine based compounds such as chlorine dioxide and/or hypochlorite, quarternary ammonium compounds, glutaraldehyde, formaldehyde, etc. By way of example, the disinfectant solution may include an aqueous hydrogen peroxide solution that contains about 5% by volume to about 20% by volume of hydrogen peroxide. More preferably, the solution contains about 8% by volume of hydrogen peroxide. In another embodiment, the disinfectant solution is an aqueous chlorine dioxide solution. For example, the disinfectant solution may be 0.2% by weight aqueous chlorine dioxide solution. More specifically, the solution may comprise 0.2 wt % oxychlorine.

As further shown in FIG. 1, the system 10 includes at least one controller 32 that controls activation and deactivation of a pump 34 and the pressurized air supply 14 to deliver the disinfectant solution and pressurized air to one or more nozzles 30 on command. An example of a controller that may be used with system 10 is an Idec-Microsmart Pentra programmable logic control. Such a controller includes RAM and ROM modules or devices and may have SD card capabilities. In an embodiment, one or more relays 27 are provided in electrical communication with the controller 32 and the one or more compressors 14.

As will be explained in more detail below, the controller 32 may be configured to control certain components in a particular room to further facilitate the delivery a disinfectant solution to the room with pressurized air. In this manner, the disinfectant solution is atomized at the nozzles 30 so the atomized solution is generally, uniformly dispersed in the room to decontaminate surfaces in the room. As will be explained in more detail below, the system 10 may include additional pumps at the nozzles 30 to deliver the disinfectant solution for injection into a respective room.

The term “controller” as used herein means a device or piece of equipment used to operate or control components of the system. By way of example, the controller may include electronic circuits and/or electronic components configured on a printed circuit board to control functions or operations of the portable system. The term “controller” is also intended to include one or more electrical relays.

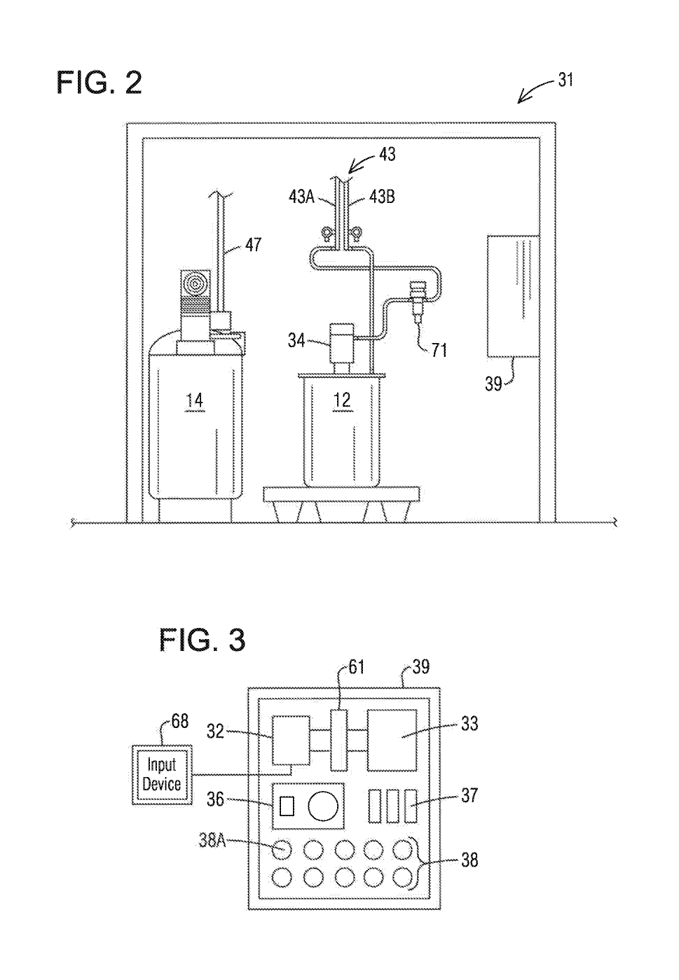

With respect to FIGS. 2, a control room 31 is illustrated including the disinfectant solution source 12 (a container holding the disinfectant solution), pressurized air supply 14 (an air compressor) and the controller 32 (which may be referred to as a first controller) including programmable logic control is mounted in a control box 39. An example of a controller that may be used with system 10 is an Idec-Microsmart Pentra programmable logic control. Such a controller includes RAM and ROM modules or devices and may have SD card capabilities. In an embodiment, one or more relays 33 are provided in electrical communication with the controller 26 and the one or more compressors 14.

The pump 34 is in fluid communication with fluid lines 43 may include a supply line 43A to the nozzles 30 and a return line 43B. The supply and fluid lines 43A, 43B may be ½ inch PVC/CPVC or chemically compatible piping and the fluid lines 43 also include ¼ inch flexible lines that connect the supply lines 43A to the nozzles. After a decontamination operation is completed any disinfectant solution remaining in any fluid lines 43 may return to the solution source 12 via return line 43B by gravity. An example of a pump that may be used with the system is a Liquidynamics 195100 which ⅓ hp, 4-10 gpm, and 20 ft maximum head pressure.

While the embodiment shown in FIG. 2 includes a single container holding the disinfectant solution, there could be multiple containers that are used with the system. Moreover, the containers of solution may be in different rooms. In addition, the containers do not have to be in the same room as the controller 32 or the pressurized air supply 14.

Air lines 47 are provided for fluid flow communication between the air compressor 14 and nozzles 30. An example of an air compressor that may be used with this system is 5-15 hp, pressure lubricated reciprocating/piston air compressor. Such air compressors are sold by Quincy Compressor located in Quincy, Ill. Air is delivered to the hub assemblies at 80 psi to 90 psi, and the below described air gauges regulate the air pressure delivered to the nozzles to about 30 psi to about 40 psi. Air lines 47 that direct flow of pressurized from the air compressor 14 to the nozzles may include 1 inch diameter aluminum main lines and ¼ inch diameter flexible lines from the main lines to the nozzles 30.

The control box 39, including the controller 32 is shown in more detail in FIG. 3. In an embodiment, 110 volts may be sufficient to power system via a power module 33 wherein the system 10, including all the component of the hub assemblies. The power module 33 may be equipped with a standard AC-DC converter to provide 24 volts to each respective hub assembly 40, 42, and 44 that are described in more detail below.

As further shown, relays 37 are provided in signal communication with the controller 32 to control the air handling system of the building. More specifically, the relays 37 are configured with the controller to turn off any air movement devices (including but not limited to air conditioning, exhaust fans, auxiliary blowers, fans) to a room during a time (injection mode) in which the disinfectant solution is delivered to a room, and to turn on the air movement devices, during a time (aeration mode) after delivery of the solution has stopped. The relays 37 may also be configured to activate and deactivate the pump 34 and air compressor 14.

At least with respect to embodiments that include a detectable disinfectant, such as hydrogen peroxide, a sensor 36 is provided to detect levels of a disinfectant associated with the solution in each respective room. In the example of a hydrogen peroxide disinfectant solution, the sensor 36 detects levels of hydrogen peroxide in each of the rooms during and after a decontamination operation. The sensor 36 is in signal communication with the controller 32, which may be programmed to send various commands or electrical signals in response to the level of disinfectant compound detected to control operations of the decontamination system 10.

An array of solenoid valves 38 is provided in fluid communication with the sensor 36 and a room in which a nozzle 30 is mounted. At least one solenoid valve 38 is associated with a corresponding room and the control room 31. One or more vacuum pumps are provided in fluid communication via air lines to draw air from a respective room or the control room 31 to sample the air to determine levels of disinfectant agent present in the room. The controller 32 is in signal communication to control the activation and deactivation of each valve 38 so the sensor 32 preferably samples only a single room at a time. The array of valves 38 may include a purge solenoid valve 38A that is activated by the controller 32 after a respective valve 38 has closed to complete a sampling. The purge valve 39A delivers ambient air to the sensor 36 to purge or clean the sensor of the disinfectant agent.

The sensor 36 is in signal communication with controller 32 to transmit signals indicative of a concentration level of the disinfectant compound in the room. The controller 32 may be programmed to initiate certain functions based on the level of the disinfectant compound detected. For example, if the concentration of the disinfectant level is at or above a predetermined level (e.g., 8 ppm of hydrogen peroxide), the controller 32 may signal a warning light (red) to be activated or remain active. If the detected concentration of the disinfectant compound drops below a predetermined level (e.g., 1 ppm) for a predetermined time duration (e.g., 3 min.), the controller may activate a light indicating the room is safe to enter. As described above, if the concentration or level of the detected disinfectant agent drops to or below a predetermined level for a predetermined time duration, the controller 32 may activate an air handling system of the building to turn on the air movement devices during an aeration or idle mode.

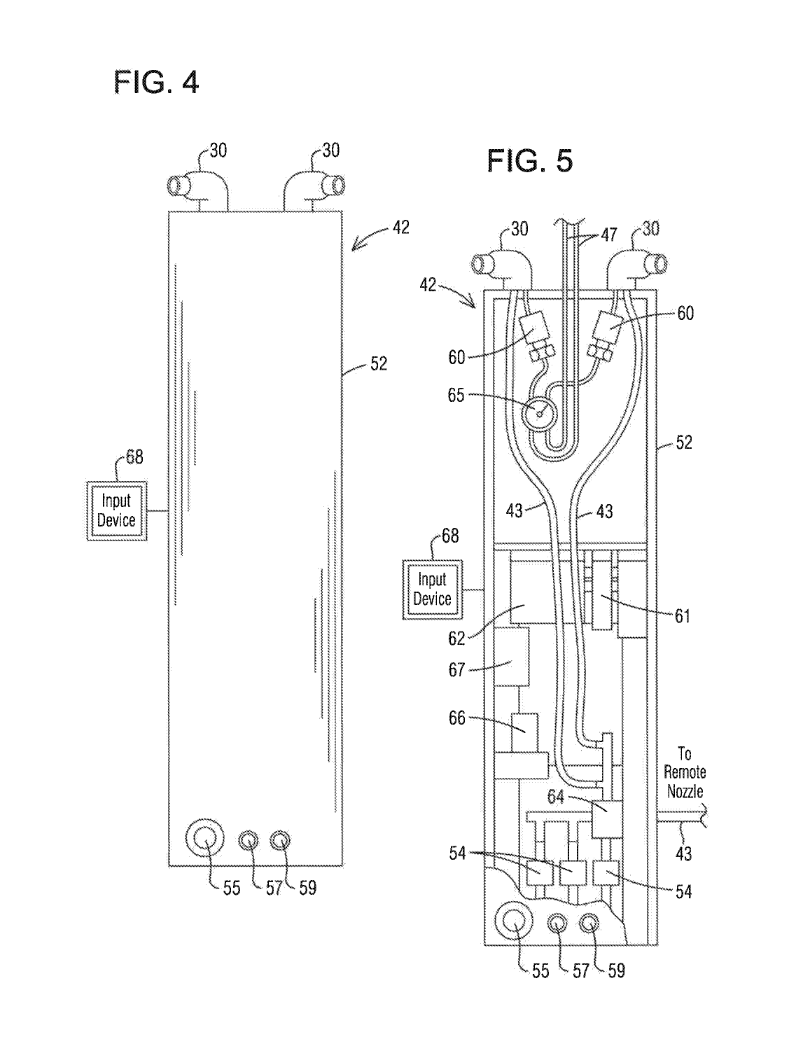

The system 10 may include solution dispensing hub assemblies (also referred to as hubs) in each of the rooms to be decontaminated. Different embodiments of dispensing hubs 40, 42, 44 and 46 are shown in FIGS. 4 through 8. The terms “hub assembly,” “dispensing hub assembly’ or “hub” as used herein means a fixture mountable on a surface of a room, preferably a wall or ceiling that includes at least one nozzle 30. As will be described below, a hub assembly may include other components such as pumps, solenoid valves, controllers, etc.

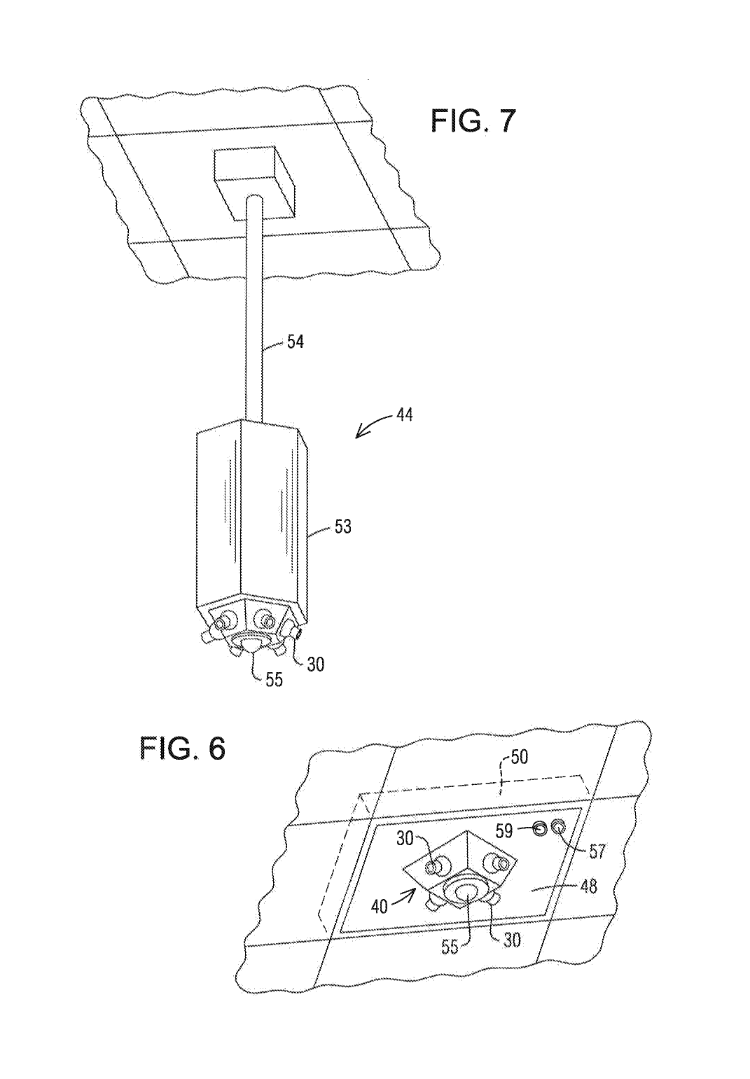

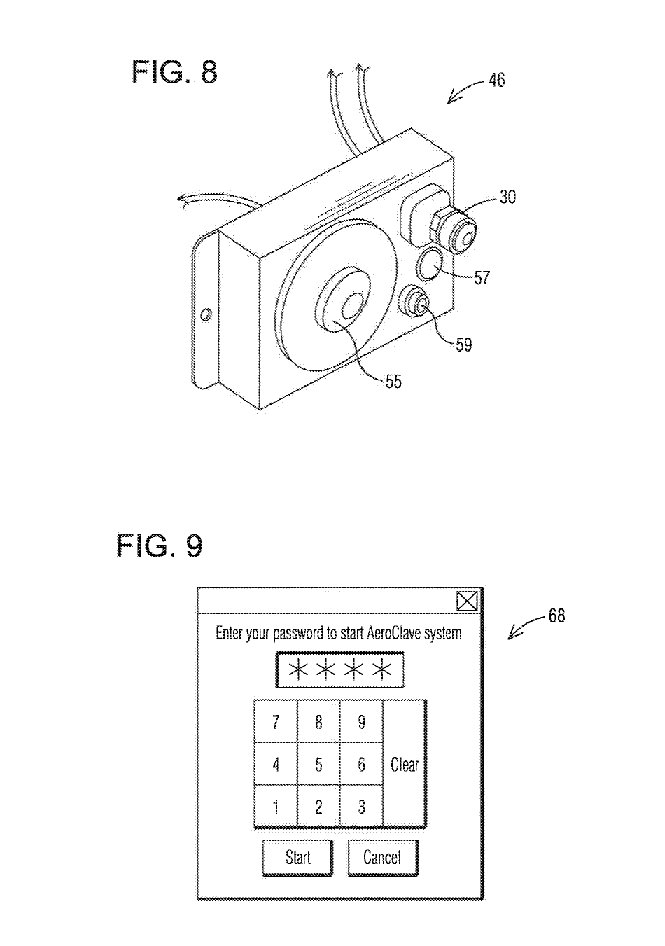

The dispensing hub 40, shown in FIG. 6, includes a mounting plate 48 that is pivotally mounted to a ceiling frame member 51. Components of the hub 40 are mounted in a housing 50 above a ceiling frame. The dispensing hub 42 of FIGS. 4 and 5 includes a housing 52, containing the below described components, mounted to a wall of a room to be decontaminated. The hub 44 shown in FIG. 7 includes housing 53 suspended from a ceiling with a pole 54 and mounting block 56. Each of the hubs 40, 42, 44 includes multiple nozzles 30, although the invention is not so limited and the hubs may include only single nozzle 30 as shown in FIG. 8, with respect to hub 46. The hub 46 may also be referred to as a remote nozzle as it does not include many of the working components of the other hubs.

The hub assembly 42, shown in FIGS. 4 and 5, is representative of the components included in the hubs 40 and 44 to perform decontamination operations. Accordingly, the description of the hub 42 also applies to the hubs 40, 44 of FIGS. 7 and 8. A power supply module 63 may be provided at the hub assembly 42 to receive power from the first controller 32 to power the components of the hub assembly 42.

As shown, the hub 42 includes one or more pumps 54 in fluid communication between the disinfectant solution source 12 and the nozzles 30. These pumps 54 may be provided in addition to pump 34 which is remote relative to the hub 44. The pump 34, in the control room 31, may be controlled to deliver disinfectant solution into fluid lines to the pumps 54, wherein the fluid line essentially serves as an in-line reservoir. The pumps 54, when activated, deliver the disinfectant to the nozzles 30. Pumps 54 are preferably peristaltic pumps that deliver disinfectant solution to the nozzles at about 30 ml/min. sold by Gardner Denver Thomas. In addition, air solenoid valves 60 and air gauges 65 control flow of pressurized air to the nozzles 30 to atomize the disinfectant solution as it is injected into the room. As described above, the gauges 65 regulate air flow to the nozzles from about 30 psi to 40 psi.

A controller 62 is provided in signal communication with the pumps 58 and valves 60 to control delivery of the disinfectant solution to the nozzles 30. To that end, a flow meter 64 is provided in signal communication with the controller 62 to monitor the flow rate of the disinfectant solution to the nozzles 30 and to monitor the amount of disinfectant solution used during a decontamination operation. Data or signals indicative of the amount of disinfectant solution used are transmitted to controller 62 and/or controller 32, one or both of which may be programmed to determine an amount of disinfectant solution that remains.

The controller 62 may be an Idec Smart Axis. The controller 62 may be configured to store data relative to decontamination operations including the amount of time a disinfectant solution is to be injected for an injection mode, the amount of disinfectant solution to be injected during the injection mode, the amount time for the below described warning phases, injection phases and dwell phases.

In addition, a sensor 66 may be provided in fluid communication with the room to detect levels of a disinfectant compound in the room during and after a decontamination operation. In an embodiment, the sensor 66 detects levels of hydrogen peroxide in the room. The sensor 66 is in signal communication with controller 62 to transmit signals indicative of a concentration level of the disinfectant compound in the room.

The controller 62 may be programmed to initiate certain functions based on the level of the disinfectant compound detected. For example, if the concentration of the disinfectant level is at or above a predetermined level (e.g., 8 ppm of hydrogen peroxide), the controller 62 may signal a warning light (red), and other safety related components (door locks, etc.) to be activated or remain active. If the detected concentration of the disinfectant compound drops below a predetermined level (e.g., 1 ppm) for a predetermined time duration (e.g., 3 min.), the controller may activate a light indicating the room is safe to enter. The above-described controller 62 may act independent of or in conjunction with the controller 32 during a decontamination operation of a room. In addition, while these hubs 40, 42 and 46 are described as including a sensor 66, such remote sensors are not necessary for the operation of the system 10.

Each of the hubs 40, 42, 44 and 46 may also include a motion detector 55 that is used to detect whether or not personnel are present in a room to be decontaminated. The function of this motion detector 55 is described in more detail relative to a decontamination operation. As further shown in FIG. 6, the hubs 40, 42 and 44, may include an air pressure gauge 65 to monitor the pressure of the air delivered to a nozzle 30. In an embodiment, air is delivered to the nozzle from about 30 psi to about 40 psi. These gauges 65 may be in signal communication with the controller 32 and/or controller 62, and the controllers 32, 62 may be programmed to terminate a decontamination operation if the air pressure drops below a determined threshold, e.g., about 30 psi, or if the air pressure meets or exceeds a threshold, e.g. about 60 psi.

With respect to the hub 46 shown in FIG. 9, a motion detector 55 is provided. In addition, this hub 46 includes a nozzle 30 for dispensing atomized disinfectant solution. An inlet port 59 provides fluid communication to the sensor 36 for monitoring levels of a disinfectant agent in the room. To that end, the other hubs 40, 42, 44 also include an inlet port 59 in fluid communication with sensors 36, 66. In addition, each hub 40, 42, 44, and 46 may include one or more warning lights 57. The hub 46, in FIG. 9, preferably does not include the other hub components such as the pumps 54, sensors 66, controllers 62, etc.

In particular hub 46 may function in conjunction with one or more of the other hubs 40, 42 or 44. For example, as shown in FIG. 5, hub assembly 42 includes three pumps 54 two of which deliver disinfectant solution to nozzles 30. One of the pumps 54 may be in fluid communication with nozzle 30 on hub 46 (FIG. 8), which for example is located in an adjacent room. The controller 62 is configured to control the third pump 54 to deliver the disinfectant solution to the nozzle 30 on hub 46.

Again referring to FIG. 1, the system may include one or more input devices 68, such as a touchscreen display that is preferably associated with each room, each second controller 62 and the first controller 32 of control room 31. Alternatively, a single input device 68 may associated with more than one room. These input devices 68 are provided in signal communication with the first controller 32 and second controllers 62 to initiate a decontamination operation, and display information related to the decontamination system or a decontamination operation.

Again referring to FIGS. 1 and 5, the system 10 may comprise a plurality of communication hubs 61, such as Ethernet hubs, associated with the first controller 32 and second controllers 62 at the hub assemblies 40, 42 and 44 mounted in the respective rooms for communication of data relative to decontamination operations taking place in the rooms. To that end the input devices 68 are on the same network. Ethernet communication cables such as an RJ-45 Ethernet cable may be incorporated for transmission of data. Such data may include the room identification information, date of decontamination, the time decontamination was initiated and completed and, the amount of disinfectant solution used during the decontamination procedure and the personnel involved in implementing the decontamination procedure. This may be particularly useful for entities such as a hospitals, police department, transit systems, airlines, etc. that may need to decontaminate multiple rooms daily, weekly or monthly.

In an embodiment of the invention the first controller 32 may be programmed to perform various functions associated with room decontamination operations. For example, the first controller 32 may be tasked to 1) coordinate the status of all the hubs, 2) present status to touchscreens, 3) operate and coordinate certain warning lights, 4) track fluid usage, and/or 5) coordinate disinfectant sensor levels from each zone, and/or 6) operate the pump 34 and air compressor 14

One of the input devices 68 may be programmed as a master input device, which can access each second controller 62 at a respective hub 40, 42, 44 to enter and store data to complete a decontamination operation. For example, data relating to a decontamination operation may include the time duration for an entire operation, the time duration for injection modes of disinfectant solutions, the amount of disinfectant solution to be injected for an injection mode, and/or time duration dwell phases between injections. Other data associated with decontamination operations may be input such as a time duration for a staging phase as described below, a specific date and time for running a decontamination operation. To that end, the data may be entered such that rooms are decontaminated sequentially or simultaneously. However, in an embodiment each controller 62 controls a decontamination operation independent of the other controllers 62 in other rooms.

Images of sample screen displays that may be used for a decontamination operation are shown in FIGS. 9-15. In order to initiate a decontamination procedure, authorized personnel may login to the system 10, preferably using a passcode at an input device 68 as seen in FIG. 9. The system 10 may further prompt the personnel to enter data relative to date, time, and room identification to initiate the decontamination procedure. It should be noted that the system 10 may be configured to perform any number of decontamination operations including decontaminating all the rooms at the same time, decontaminating multiple rooms sequentially, or decontaminate only a single room. Accordingly, the above-described input devices 68 may be dedicated to a single room or multiple rooms to initiate decontamination procedures as necessary.

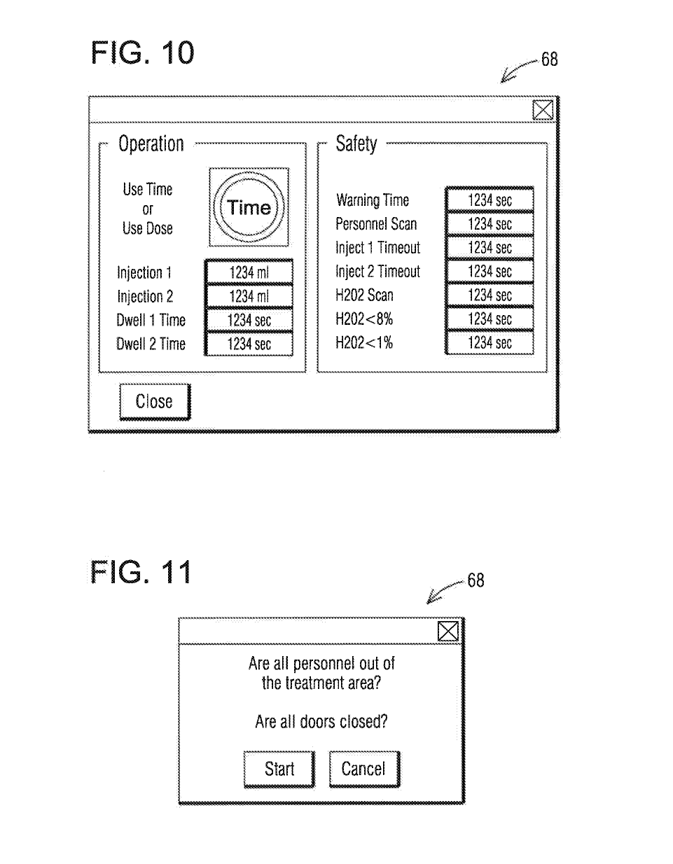

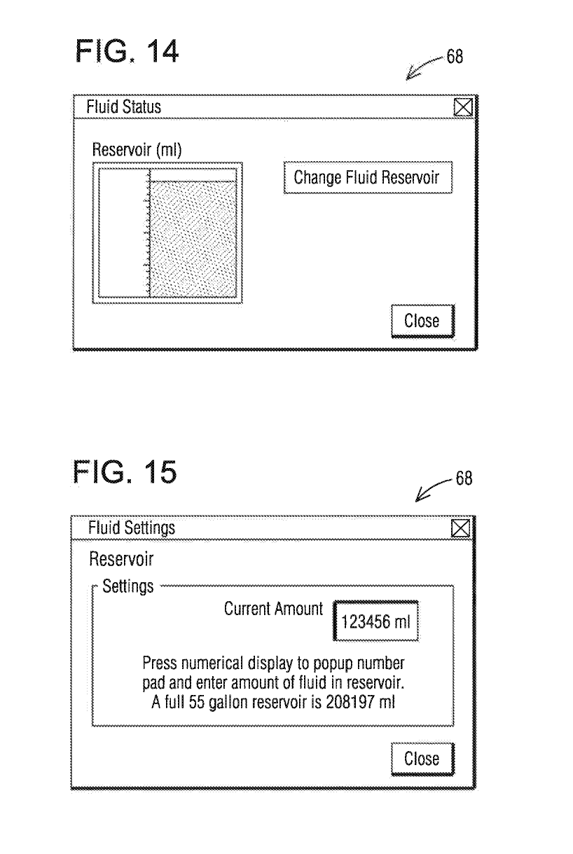

In addition, the screen display may prompt the personnel to select a mode of operation, namely “Time” or “Dose.” For a “Time” mode the total injection time is entered and in the “Dose” mode the total volume of disinfectant solution to be used is entered. As shown in FIG. 10, the screen of input device 68 will display data related to the different phases of the decontamination operation, at which time the personnel may choose to change any of the listed variables. After entry of decontamination procedure data, a “START” button or icon (FIG. 14) will appear and authorized personnel presses the button or icon.

At this time, a “warning mode” or “staging phase” is initiated for a first time duration during which a visual and/or auditory warning signal is started for personnel to exit the room. As shown in FIG. 13 this warning signal may include a yellow light or indicator at the display of the input device. The controller 32 is configured with a time delay wherein after a preset time duration during which the motion detector 55 detects no motion, i.e. no personnel in the interior space to be decontaminated, the controller 32 initiates certain operations. More specifically, the controller 32 is linked to the buildings internal communication or computer system to determine if certain building functions are performed. For example, the controller 32 may transmit one or more control signals to lock the doors of a room and turn off HVAC functions, etc. To that end, the building communication system may transmit signals to the controller 32 indicative of the status of these building functions.

As indicated above, these functions may be performed during a first time period that may be referred to as a warning mode or staging phase; and, a warning light or strobe and/or an audible signal may be activated such as the light 57 on any one of the hub assemblies described above. However, if motion is detected by the motion detector 55, during this staging phase or first time duration, the first or second controller 32, 62 will not initiate the remaining operations associated with the staging phase, or otherwise proceed unless no motion is detected in the room for a predetermined time. If no motion is detected for this predetermined time then the building operations may be completed.

Once the first time duration (warning mode or staging phase) has elapsed and the building operations described above are completed, the controller 32 activates the pumps 34 and air compressors 14, and the controller 62 activates pumps 54 to deliver the disinfectant solution and compressed air to the nozzles 30 to atomize the solution and inject the same into a room. The time duration of this injection mode may vary according to the volume of the room and the volume of disinfectant solution need to decontaminate the space.

To that end, as provided in FIG. 10, the injection mode may be divided into discrete timed periods including one or more dwell times during which no disinfectant solution is injected interposed between two or more injection periods. By way of example, an injection may be made continuously for 4 minutes for a first injection period followed by a 2 minute first dwell time, which is then followed by a second 4 minute injection period followed by second 2 minute dwell time for an injection phase lasting 12 minutes. During the injection mode, and as represented in legend of FIG. 13, a red warning signal may be displayed indicating the building/room is undergoing decontamination.

After the injection mode has been completed, an aeration mode (delete/shut down mode) is initiated by the controller 32 transmitting one or more signals to activate the building HVAC system. Note, in an embodiment the warning lights and audible signals may remain on during the staging, injection and aeration modes. During the aeration mode, the sensor 36 detects levels of sanitizing compound within a room. The controller 32 may be programmed such that once the level of the detected disinfectant compound has dropped to a predetermined threshold for a pre-determined time duration, signals are generated to turn on the HVAC and unlock doors to the room. For example, if hydrogen peroxide is the detected compound the threshold concentration may be 1 ppm and the time duration may be 3 minutes. In addition, the warning signals may be deactivated or changed to indicate that it is safe for authorized personnel to enter the interior space and decontamination has been completed. As shown in FIG. 10, a green light may indicate the interior space and building is safe for personnel to enter

As further shown in FIG. 14, the controller 32 and input device 68, with a monitor or display screen, may be configured to display, when a decontamination operation is completed, an amount of disinfectant solution used and an amount of disinfectant solution remaining in the reservoir 12, With respect to FIG. 15, to the extent that disinfectant solution is added to the reservoir 12, the amount of disinfectant solution in the reservoir 12 may be updated by inputting the data at the touchscreen.

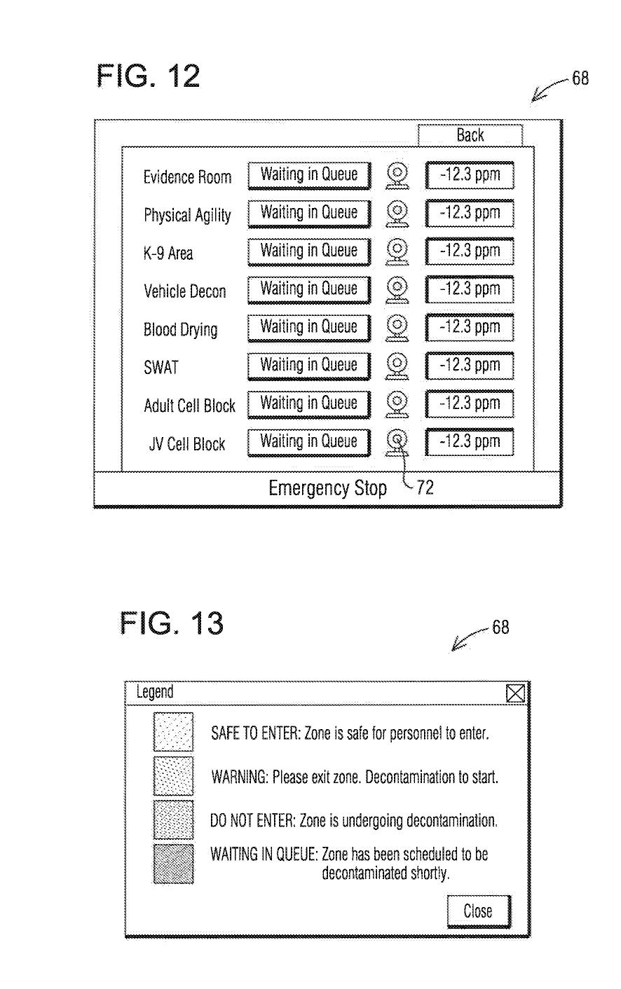

In reference to FIG. 12, the input device 68 has been prompted to display a status of each of the different rooms or zones. As shown, symbols 72 may be color indicative of the status of a room, or each room. The color indication is preferably consistent with the legend of FIG. 13. Such a screen may be accessed any time before, during or after a decontamination operation.

This decontamination data may be stored on a server database that is remotely accessible for example via a secure Internet website. The system 10, including a server database, may be linked with a wired/wireless/cellular communications network, so that the website can be accessed via a personal computer or hand-held personal devices such as smartphones, I-pads, etc. Accordingly, personnel using the system or a decontamination service company can remotely access data in the database to determine dates and times decontamination operations have taken place, the amount of disinfectant solution remaining in the system, identify personnel that have accessed the system for running decontamination operations, etc.

While certain embodiments of the present invention have been shown and described herein, such embodiments are provided by way of example only. Numerous variations, changes and substitutions will occur to those of skill in the art without departing from the invention herein. Accordingly, it is intended that the invention be limited only by the spirit and scope of the appended claims.

What is claimed is:

1. A decontamination system for a building, comprising:
a disinfectant solution source with a disinfectant solution;
a plurality of nozzles in fluid communication with the disinfectant solution source and each nozzle is associated with a room in the building;
one or more pumps in fluid communication with each nozzle and the disinfectant solution source;
a pressurized air supply in fluid communication with each nozzle;
a plurality of fluid lines connecting the disinfectant solution source to the nozzles;
a plurality of air lines connecting the pressurized air supply to the nozzles; and,
one or more controllers in signal communication with the one or more pumps and the pressurized air supply to activate the pump(s) and pressurized air supply to simultaneously deliver the disinfectant solution and pressurized air to one or more of the nozzles in a respective room.

2. The decontamination system of claim 1, further comprising one or more motion detectors in a room having a nozzle and the one or more controllers is configured to activate the one or more pumps to deliver disinfectant solution to the nozzles after a time duration during which the motion detector does not detect movement in the room.

3. The decontamination system of claim 2, wherein the motion detector and nozzle are mounted to a hub assembly.

4. The decontamination system of claim 1, further comprising a first pump in a first room and the first pump is operatively connected to the disinfectant solution source and in signal communication with the one or more controllers to deliver the disinfectant solution into the fluid lines and at least one second pump in a second room and the second pump is in fluid communication with the first pump and fluid lines to deliver the disinfectant solution to a nozzle in the second room.

5. The decontamination system of claim 4, further comprising one or motion detectors in the second room and the one or more controllers is configured to activate the first pump and the at least one second pump to deliver disinfectant solution to the nozzle after a time duration during which the motion detector does not detect movement in the room.

6. The decontamination system of claim 4, wherein the one or more controllers includes a first controller and a second controller wherein the second controller is in the second room, and an input device is associated with the first controller or second controller to input parameters associated with a decontamination operation and the first controller is configured, on command, to activate the first pump and the pressurized air supply, and the second controller is configured to activate the at least one second pump.

7. The decontamination system of claim 1, wherein the one or more controllers is further configured to initiate building functions related to a room to be decontaminated before and/or after disinfectant solution is delivered to the room.

8. The decontamination system of claim 7, wherein the building functions include locking and/or unlocking a door to a room to be decontaminated and/or turning on and/or off HVAC functions to the room to be decontaminated.

9. The decontamination system of claim 1, further comprising one or more sensors in fluid communication with a room to be decontaminated for monitoring levels of an active disinfectant of the disinfectant solution and the sensor is in signal communication with the one or more controllers transmitting signals indicative of a level of a disinfectant in a room.

10. The decontamination system of claim 9, further comprising an air inlet port in a room with a nozzle and an airline through which air is delivered from the respective room through the air inlet port to the sensor.

11. The decontamination system of claim 10, wherein the sensor is in signal communication with the one or more controllers to transmit signals indicative of a level of the disinfectant in the room and the controller is configured to generate a signal to an audible or visual indicator when the disinfectant is above or below a threshold concentration.

12. The decontamination system of claim 11, further comprising an array of solenoid valves in signal communication with the one more controllers and each solenoid valve is associated with a room with a nozzle and air inlet port and the one or more controllers is configured to sequentially activate the solenoid valves to deliver air from the rooms to detect levels of a disinfectant in respective room.

13. The decontamination system of claim 12, wherein the array of solenoid valves includes a valve that is activated by the controller to deliver ambient air to the sensor after a room has been decontaminated and after the controller and sensor have determined that the level of disinfectant has dropped to or below a threshold concentration for a time duration.

14. The decontamination system of claim 1, wherein the one or more controllers includes a first controller in a first room and a second controller in a second room with a pump and a nozzle in the second room and the first controller is configured to activate the second controller to activate the pump in the second room.

15. The decontamination system of claim 1, wherein a first nozzle is in a first room and a second nozzle is in a second room, and the one or pumps includes at least two pumps in the first room wherein one pump delivers disinfectant solution to a nozzle in the first room and another pump delivers disinfectant solution to second nozzle in the second room.

16. A decontamination system for a building, comprising:
a disinfectant solution source with a disinfectant solution stored in a first room of the building;
at least one nozzle positioned in a second room of the building and the at least one nozzle is in fluid communication with the disinfectant solution source;
one or more pumps in fluid communication with each nozzle and the disinfectant solution source;
a pressurized air supply in fluid communication with each nozzle;
a plurality of fluid lines connecting the disinfectant solution source to each nozzle;
a plurality of air lines connecting the pressurized air supply to each nozzle; and,
one or more controllers in signal communication with the one or more pumps and the pressurized air supply to activate the pump(s) and pressurized air supply to simultaneously deliver the disinfectant solution and pressurized air to one or more of the nozzles in a respective room.

17. The decontamination system of claim 16, wherein the one or more pumps includes a first pump in fluid communication with the disinfectant source that delivers the disinfectant into fluid lines leading to a second pump and at least one nozzle in the second room and the second pump delivers the disinfectant solution to the at least one nozzle.

18. The decontamination system of claim 17, wherein the system includes at least two second pumps in the second room wherein one of the second pumps delivers disinfectant solution to a nozzle second room and the other second pump delivers disinfectant solution to a nozzle in a third room.

19. The decontamination system of claim 17, wherein the one or more controllers includes a first controller that controls activation of the first pump, pressurized air supply and at least one second controller associated with the second room and the second controller is configured to activate the second pump.

20. The decontamination system of claim 16, further comprising one or motion detectors in the second room having the at least one nozzle and the one or more controllers is configured to activate the one or more pumps to deliver disinfectant solution to the nozzles after a time duration during which the motion detector does not detect movement in the room.

21. The decontamination system of claim 16, wherein the one or more controllers is configured to initiate building functions before or after a decontamination operation including locking and/or unlocking a door to a room to be decontaminated and/or turning on and/or off HVAC functions to the room to be decontaminated.
